(19) United States
(12) Patent Application Publication
(10) Pub. No.: US 2021/0366078 A1
(43) Pub. Date: Nov. 25, 2021

(54) IMAGE PROCESSING DEVICE, IMAGE PROCESSING METHOD, AND IMAGE PROCESSING SYSTEM

(71) Applicant: PANASONIC INTELLECTUAL PROPERTY MANAGEMENT CO., LTD., Osaka (JP)

(72) Inventors: Tadanori TEZUKA, Fukuoka (JP); Tsuyoshi NAKAMURA, Fukuoka (JP)

(73) Assignee: PANASONIC INTELLECTUAL PROPERTY MANAGEMENT CO., LTD., Osaka (JP)

(21) Appl. No.: 17/392,639

(22) Filed: Aug. 3, 2021

Related U.S. Application Data
(63) Continuation of application No. PCT/JP2020/003236, filed on Jan. 29, 2020.

(30) Foreign Application Priority Data
Feb. 6, 2019 (JP) 2019-019740

(51) Int. Cl. G06T 3/40; G06T 7/20

(52) U.S. Cl. CPC G06T 3/40; G06T 7/20

(57) ABSTRACT

An image processing device includes a memory that stores instructions, and a processor that, when executing the instructions stored in the memory, performs a process. The process includes: averaging an input image in units of N×M pixels (N, M: an integer of 2 or larger) in a spatial direction for each grid composed of one pixel or a plurality of pixels, the input image being composed of (S×T) pixels (S, T: a positive integer) having an information amount of a (a: a power of 2) bits per pixel, and defining an averaging result in units of N×M pixels for each pixel or grid by an information amount of (a+b) bits per pixel (b: an integer of 2 or larger) and generating a reduced image composed of (S×T)/(N×M) pixels having the information amount of (a+b) bits per pixel. A value of b is an exponent c (c: a positive integer) of a power value of 2 close to (N×M), or (c+1).

This is a continuation of International Application No. PCT/JP2020/003236 filed on Jan. 29, 2020, and claims priority from Japanese Patent Application No. 2019-019740 filed on Feb. 6, 2019, the entire content of which is incorporated herein by reference.

### TECHNICAL FIELD

The present disclosure relates to an image processing device that processes an input image, an image processing method, and an image processing system.

### BACKGROUND ART

JP-A-2011-259325 discloses a moving image encoding device that generates a predicted image based on a reference image and a block of interest of an image to be encoded, obtains an error image from the predicted image and the block of interest, generates a locally decoded image based on the error image and the predicted image, obtains a difference between the locally decoded image and the block of interest and compresses the difference to generate a compressed difference image, and writes the compressed difference image in a memory. According to the moving image encoding device, an amount of data to be written to the memory in order to use the locally decoded image can be reduced.

However, in a configuration according to JP-A-2011-259325, data of the difference image created to obtain the difference between the locally decoded image and the block of interest is rounded by fraction processing (that is, lower bits are truncated). Since JP-A-2011-259325 aims to reduce the amount of data of the compressed difference image transferred to a frame memory unit, the lower bits of the data of the difference image used for generating the compressed difference image are truncated. Therefore, even if an attempt is made to sense, using an image compressed by the moving image encoding device, presence or absence of a feature such as motion information or biological information of an object in the image, there is a high possibility that detection of the motion information or the biological information becomes difficult by the above-described fraction processing (that is, rounding processing), and there is a problem that appropriate sensing becomes difficult.

### SUMMARY

An object of the present disclosure is to provide an image processing device, an image processing method and an image processing system capable of effectively compressing an input image to reduce a data size while preventing deterioration in detection accuracy of presence or absence of motion information or biological information of an object in the compressed image.

Aspect of non-limiting embodiments of the present disclosure relates to provide an image processing device including: an averaging processing unit that averages an input image in units of N×M pixels (N, M: an integer of 2 or larger) in a spatial direction for each grid composed of one pixel or a plurality of pixels, the input image being composed of (S×T) pixels (S, T: a positive integer) having an information amount of a (a: a power of 2) bits per pixel; and a generating unit that defines an averaging result in units of N×M pixels for each pixel or grid by an information amount of (a+b) bits per pixel (b: an integer of 2 or larger) and generates a reduced image composed of (S×T)/(N×M) pixels having the information amount of (a+b) bits per pixel. A value of b is an exponent c (c: a positive integer) of a power value of 2 close to (N×M), or (c+1).

In addition, another aspect of non-limiting embodiments of the present disclosure relates to provide an image processing method in an image processing device, the image processing method including: a step of averaging an input image in units of N×M pixels (N, M: an integer of 2 or larger) in a spatial direction for each grid composed of one pixel or a plurality of pixels, the input image being composed of (S×T) pixels (S, T: a positive integer) having an information amount of a (a: a power of 2) bits per pixel; and a step of defining an averaging result in units of N×M pixels for each pixel or grid by an information amount of (a+b) bits per pixel (b: an integer of 2 or larger) and generating a reduced image composed of (S×T)/(N×M) pixels having the information amount of (a+b) bits per pixel. A value of b is an exponent c (c: a positive integer) of a power value of 2 close to (N×M), or (c+1).

Further, another aspect of non-limiting embodiments of the present disclosure relates to provide an image processing system in which an image processing device and a sensing device are connected so as to communicate with each other. The image processing device averages an input image in units of N×M pixels (N, M: an integer of 2 or larger) in a spatial direction for each grid composed of one pixel or a plurality of pixels, the input image being composed of (S×T) pixels (S, T: a positive integer) having an information amount of a (a: a power of 2) bits per pixel, and defines an averaging result in units of N×M pixels for each pixel or grid by an information amount of (a+b) bits per pixel (b: an integer of 2 or larger), generates a reduced image composed of (S×T)/(N×M) pixels having the information amount of (a+b) bits per pixel, and sends the reduced image to the sensing device. The sensing device senses motion information or biological information of an object using the reduced image sent from the image processing device. A value of b is an exponent c (c: a positive integer) of a power value of 2 close to (N×M), or (c+1).

According to the present disclosure, it is possible to effectively compress an input image to reduce a data size while preventing deterioration in detection accuracy of presence or absence of motion information or biological information of an object in the compressed image.

### BRIEF DESCRIPTION OF DRAWINGS

Exemplary embodiments of the present disclosure will be described in detail based on the following figures.

FIG. 1 is a diagram showing a configuration example of an image processing system according to an embodiment.

FIG. 2 is a diagram showing an outline of an operation of the image processing system.

FIG. 3 is a view showing an example of each of an input image and a reduced image.

FIG. 4 is a diagram explaining image compression by pixel addition and averaging.

FIG. 5 is a diagram explaining pixel addition and averaging of 8×8 pixels performed on an input image.

FIG. 6 is a diagram showing registered contents of an addition and averaging pixel number table.

FIG. 7 is a diagram showing using the reduced image timings of reduced images.

FIG. 8 is a graph showing pixel value data of the input image.

FIG. 9 is a graph showing the pixel value data on which rounding processing is not performed and the pixel value data on which the rounding processing is performed in the pixel addition and averaging.

FIG. 10 is a diagram explaining an effective component of a pixel signal when the pixel addition and averaging is performed without the rounding processing.

FIG. 11 is a graph showing image value data after the pixel addition and averaging with the rounding processing and the pixel value data after the pixel addition and averaging without the rounding processing according to a first embodiment in each of Comparative Example 1, Comparative Example 2 and Comparative Example 3.

FIG. 12 is a flowchart showing a sensing operation procedure of an image processing system according to the first embodiment.

FIG. 13 is a flowchart showing an image reduction processing procedure in step S2.

FIG. 14 is a flowchart showing a grid unit reduction processing procedure in step S12.

FIG. 15 is a diagram showing registered contents of a specific size selection table indicating a specific size corresponding to a sensing target.

FIG. 16 is a flowchart showing a sensing operation procedure of an image processing system according to a first modification of the first embodiment.

FIG. 17 is a flowchart showing a procedure for generating reduced images in a plurality of sizes in step S2A.

FIG. 18 is a diagram showing a configuration of an integrated sensing device.

### DESCRIPTION OF EMBODIMENTS

Hereinafter, an embodiment specifically disclosing configurations and operations of an image processing device, an image processing method and an image processing system according to the present disclosure will be described in detail with reference to the drawings as appropriate. However, unnecessarily detailed description may be omitted. For example, detailed description of a well-known matter or repeated description of substantially the same configuration may be omitted. This is to avoid unnecessary redundancy in the following description and to facilitate understanding of those skilled in the art. The accompanying drawings and the following description are provided for those skilled in the art to fully understand the present disclosure, and are not intended to limit a subject matter described in the claims.

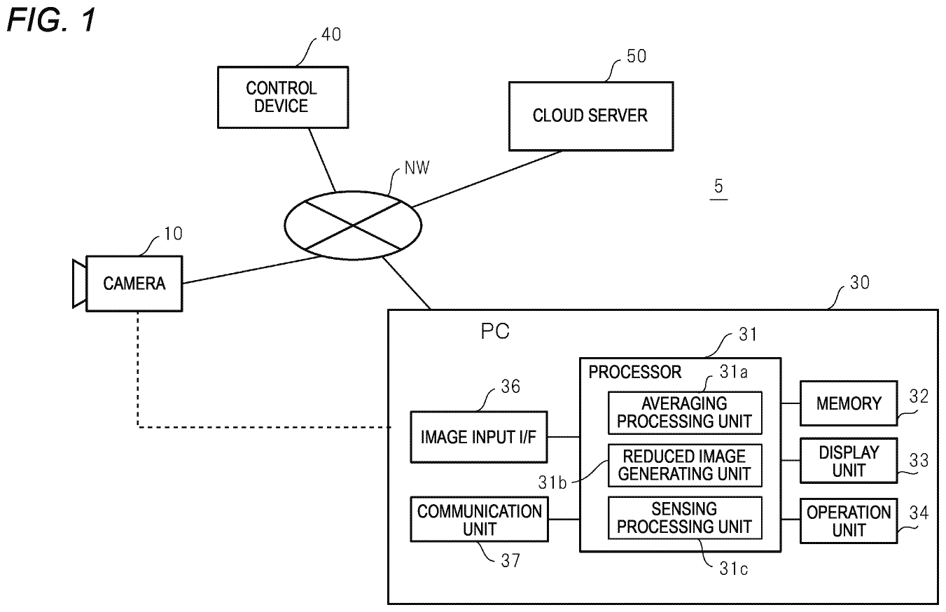

FIG. 1 is a diagram showing a configuration example of an image processing system 5 according to the present embodiment. The image processing system 5 includes a camera 10, a personal computer (PC) 30, a control device 40 and a cloud server 50. The camera 10, the PC 30, the control device 40 and the cloud server 50 are connected to a network NW and can communicate with each other. The camera 10 may be directly connected to the PC 30 in a wired or wireless manner, or may be integrally provided in the PC 30.

In the image processing system 5, the PC 30 or the cloud server 50 compresses each frame image constituting the moving image captured by the camera 10 for sensing performed by the control device 40 (refer to the following description) to reduce a data amount of the moving image. Accordingly, a communication amount (a traffic amount) of data of the network NW can be reduced. At this time, the PC 30 or the cloud server 50 compresses data of the moving image input from camera 10 while reducing the data in a spatial direction (that is, vertical and horizontal sizes) and maintaining motion information or biological information of a subject in the moving image without reducing the motion information or the biological information in a time direction. The PC 30 or the cloud server 50 performs, for example, the sensing of the frame images constituting the captured moving image, and controls an operation of the control device 40 based on sensing information corresponding to the sensing result (refer to the following description).

The camera 10 captures an image of a subject serving as a sensing target. The sensing target is biological information (hereinafter, may be referred to as “vital information”) of the subject (for example, a person), a minute motion of the subject, a short-term motion in the time direction, or a long-term motion in the time direction. Examples of the vital information of the subject include presence or absence of a person, a pulse and a heart rate fluctuation. Examples of the minute motion of the subject include a slight body motion and a respiratory motion. Examples of the short-term motion of the subject include a motion and shaking of a person or an object. Examples of the long-term motion of the subject include a flow line, an arrangement of an object such as furniture, daylighting (sunlight, ray of weathering sun), and a position of an entrance or a window.

The camera 10 includes a solid-state imaging element (that is, an image sensor) such as a charge-coupled device (CCD) or a complementary metal oxide semiconductor (CMOS), forms an image of light from a subject, converts the formed optical image into an electric signal, and outputs a video signal. The video signal output from the camera 10 is input to the PC 30 as moving image data. The number of cameras 2 is not limited to one, and may be plural. The camera 10 may be an infrared camera capable of emitting near infrared light and receiving the reflected light. The camera 10 may be a fixed camera, or may be a pan tilt zoom (PTZ) camera capable of pan, tilt and zoom. The camera 10 is an example of a sensing device. The sensing device may be, in addition to a camera, a thermography, a scanner or the like capable of acquiring a captured image of a subject.

The PC 30 as an example of the image processing device compresses the captured image (the above-described frame images) input from the camera 10 to generate a reduced image. Hereinafter, the captured image input from the camera 10 may be referred to as an “input image”. The PC 30 may input a moving image or a captured image accumulated in the cloud server 50 instead of inputting the captured image from the camera 10. The PC 30 includes a processor 31, a memory 32, a display unit 33, an operation unit 34, an image input interface 36 and a communication unit 37. In FIG. 1, the interface is abbreviated as “I/F” for convenience.

The processor 31 controls an operation of each unit of the PC 30, and is configured using a central processing unit (CPU), a digital signal processor (DSP), a field programmable gate array (FPGA) or the like. The processor 31 controls the operation of each unit of the PC 30. The processor 31 functions as a control unit of the PC 30, and performs control processing for controlling the operation of each unit of the PC 30 as a whole, data input/output processing with respect to each unit of the PC 30, data calculation processing, and data storage processing. The processor 31 operates according to execution of a program stored in a ROM in the memory 32.

The processor 31 includes an averaging processing unit 31a that averages an input image from the camera 10 in units of N×M pixels (N, M: an integer of 2 or larger) in the spatial direction, a reduced image generating unit 31b that generates a reduced image based on an averaging result in units of N×M pixels, and a sensing processing unit 31c that senses motion information or biological information of an object using the reduced image. The averaging processing unit 31a, the reduced image generating unit 31b and the sensing processing unit 31c are realized as functional configurations when the processor 31 executes a program stored in advance in the memory 32. The sensing processing unit 31c may be configured by executing the program at the cloud server 50.

The memory 32 stores the moving image data such as the input image, various types of calculation data, programs, and the like. The memory 32 includes a primary storage device (for example, a random access memory (RAM) or a read only memory (ROM)). The memory 32 may include a secondary storage device (for example, a hard disk drive (HDD) or a solid state drive (SSD)) or a tertiary storage device (for example, an optical disk or an SD card).

The display unit 33 displays a moving image, a reduced image, a sensing result and the like. The display unit 33 includes a liquid crystal display device, an organic electroluminescence (EL) device or another display device.

The operation unit 34 receives input of various types of data and information from a user. The operation unit 34 includes a mouse, a keyboard, a touch pad, a touch panel, a microphone or other input devices.

When the camera 10 is directly connected to the PC 30, the image input interface 36 inputs image data (data including a moving image or a still image) captured by the camera 10. The image input interface 36 includes an interface capable of wired connection, such as a high-definition multimedia interface (HDMI) (registered trademark) or a universal serial bus (USB) type-C capable of transferring image data at high speed. When the camera 10 is wirelessly connected, the image input interface 36 includes an interface such as short-range wireless communication (for example, Bluetooth (registered trademark) communication).

The communication unit 37 communicates with other devices connected to the network NW in a wireless or wired manner, and transmits and receives data such as image data and various calculation results. Examples of a communication method may include communication methods such as a wide area network (WAN), a local area network (LAN), power line communication, short-range wireless communication (for example, Bluetooth (registered trademark) communication), and communication for a mobile phone.

The control device 40 is a device that is controlled according to an instruction from the PC 30 or the cloud server 50. Examples of the control device 40 include an air conditioner capable of changing a wind direction, an air volume and the like, and a light capable of adjusting an illumination position, an amount of light and the like.

The cloud server 50 as an example of a sensing device includes a processor, a memory, a storage and a communication unit (none of which are shown), has a function of compressing an input image to generate a reduced image and a function of sensing motion information or biological information of an object using the reduced image, and can input image data from a large number of cameras 10 connected to the network NW, similarly to the PC 30.

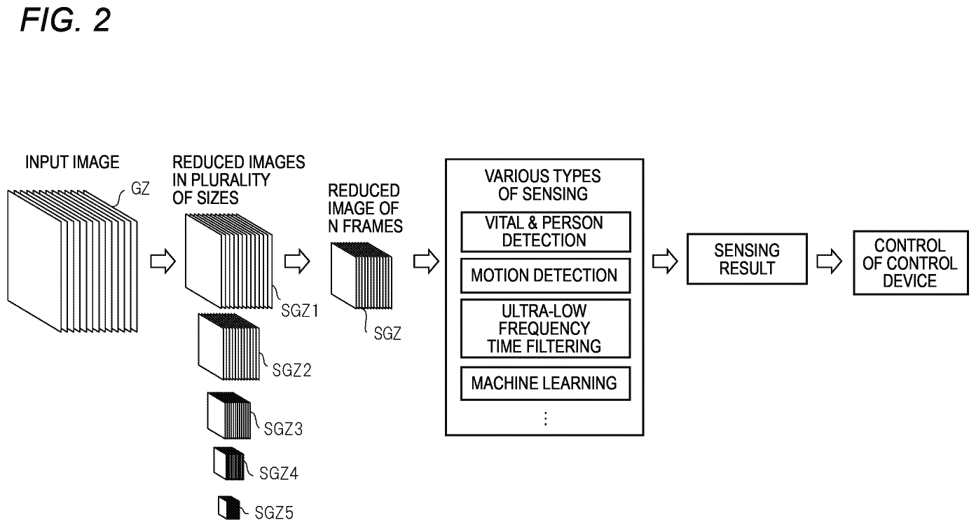

FIG. 2 is a diagram showing an outline of an operation of the image processing system 5. The main operation of the image processing system 5 described below may be performed by either the PC 30 as the example of the image processing device or the cloud server 50. In general, when an amount of data processing is small, the PC 30 serving as an edge terminal may execute the processing, and when the amount of data processing is large, the cloud server 50 may execute the processing. Here, in order to make the description easy to understand, a case where the PC 30 mainly executes the processing is shown.

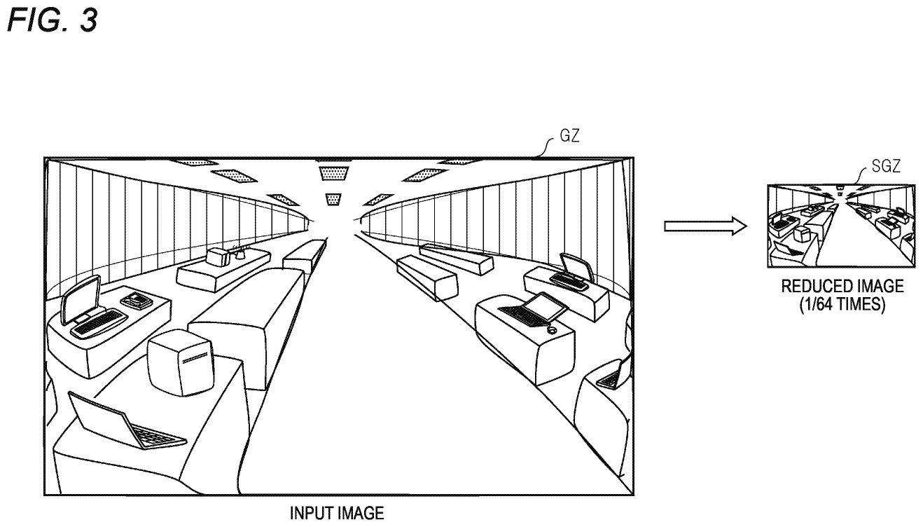

The camera 10 captures an image of a subject such as an office (see FIG. 3), and outputs or transmits the captured moving image to the PC 30. The PC 30 acquires each frame image included in the input image from the camera 10 as an input image GZ. A data size of such an input image GZ tends to increase as image quality is higher in a high definition (HD) class such as 4K or 8K.

The PC 30 compresses the input image GZ, which is an original image before compression, and generates and obtains reduced images SGZ having a plurality of types of data sizes (see below). During this image compression, the PC 30 performs different types of pixel addition and averaging processing (an example of averaging processing) of, for example, 8×8 pixels, 16×16 pixels, 32×32 pixels, 64×64 pixels and 128×128 pixels on the input image GZ, and obtains reduced images SGZ1 to SGZ5 (see FIG. 2). When all of these types of pixel addition and averaging are performed, an information amount (a data size) is compressed to an information amount (a data size) of about 8% of the input image GZ that is the original image. Therefore, a data amount corresponding to 12 frames of each of the reduced images SGZ1 to SGZ5 is the same as a data amount corresponding to frames of the input image GZ1 that is the original image. When the other types of pixel addition and averaging (that is, 16×16 pixels, 32×32 pixels, 64×64 pixels and 128×128 pixels) excluding the pixel addition and averaging of 8×8 pixels are performed, the information amount (the data size) is compressed to an information amount (a data size) of about 2% of the input image GZ that is the original image. Therefore, a data amount corresponding to 50 frames of each of the reduced images SGZ2 to SGZ5 is the same as the data amount corresponding to the frames of the input image GZ1 that is the original image.

The PC 30 performs sensing based on the reduced images SGZ of N (N is any natural number) frames accumulated in the time direction. In the sensing, pulse detection, person position detection processing and motion detection processing are performed as examples of vital information of the subject (for example, a person). In the PC 30, ultra-low frequency time filtering processing, machine learning and the like may be performed. The PC 30 controls the operation of the control device 40 based on a sensing result. For example, when the control device 40 is an air conditioner, the PC 30 instructs the air conditioner to change a direction, an air volume and the like of air blown out from the air conditioner.

FIG. 3 is a view showing an example of each of the input image GZ and the reduced image SGZ. The input image GZ is the original image captured by the camera 10 and is for example, an image captured in the office and before being compressed in. The reduced image SGZ is, for example, a reduced image obtained by performing pixel addition and averaging of 8×8 pixels on the input image GZ by the PC 30. In the input image GZ, a situation in the office is clearly displayed. In the office, there are motions such as a motion of a person. On the other hand, in the reduced image SGZ, image quality indicating the situation in the office is displayed in a degraded state, but it is suitable for sensing since motion information such as the motion of the person is retained.

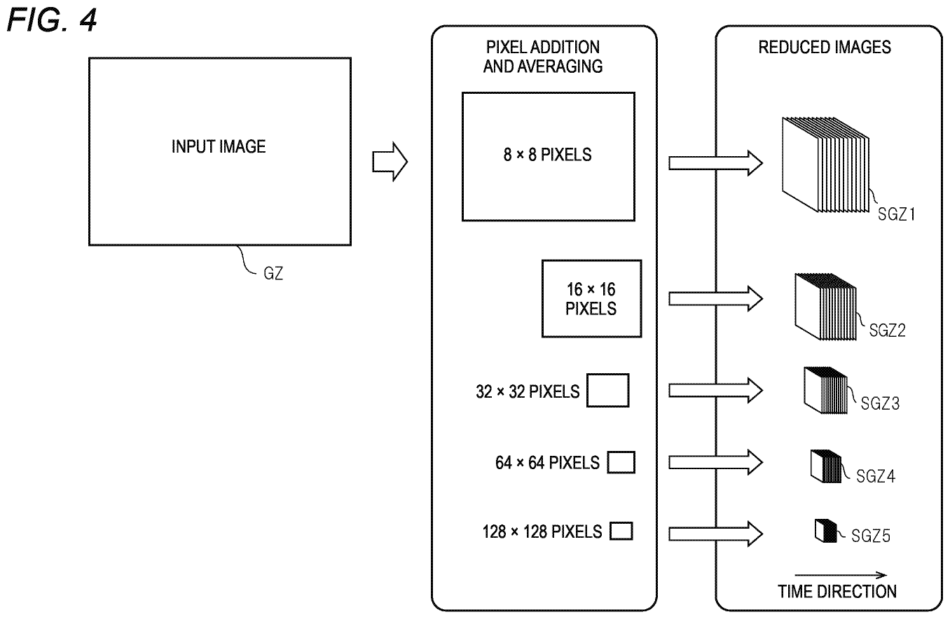

FIG. 4 is a diagram explaining image compression by pixel addition and averaging. During the image compression, the PC 30 performs pixel addition and averaging of, for example, 8×8 pixels, 16×16 pixels, 32×32 pixels, 64×64 pixels and 128×128 pixels on the input image GZ without performing rounding processing (in other words, integer conversion processing of rounding off fractions after the decimal point), and obtains reduced images SGZ1, SGZ2, SGZ3, SGZ4, SGZ5, respectively. When performing the pixel addition and averaging, the PC 30 holds a value after the decimal point as a pixel value. When the value after the decimal point is held, an image value is expressed in, for example, a single-precision floating-point format. Here, a minute change in the input image is likely to appear in the value after the decimal point of the pixel value. Therefore, the PC 30 holds the value after the decimal point as the pixel value after the pixel addition and averaging, so that the minute change of the subject existing in the input image that is the original image can be captured even during the compression.

When the pixel addition and averaging of 8×8 pixels, 16×16 pixels, 32×32 pixels, 64×64 pixels and 128×128 pixels is performed, these reduced images are compressed to the data amount of about 8% of the original image as described above. When sensing processing is performed using these reduced images, the PC 30 can reduce an amount of calculation required for the sensing processing. Therefore, the PC 30 can perform the sensing processing in real time.

The PC 30 may perform any one or more types of pixel addition and averaging without performing all of the five types of pixel addition and averaging. When any one or more types of pixel addition and averaging are performed, the PC 30 may select the pixel addition and averaging according to a sensing target. For example, the addition and averaging of 8×8 pixels may be used for the motion detection or the person detection. The addition and averaging of 64×64 pixels and 128×128 pixels may be used for the pulse detection that is the vital information. All of the five types of pixel addition and averaging may be used for long time motion detection, for example, slow shake detection. In this way, in a case of limiting to one or more types of pixel addition and averaging, a compression ratio of the data amount is higher than that in a case of performing all types of pixel addition and averaging. The PC 30 can significantly reduce the amount of calculation required for the sensing processing.

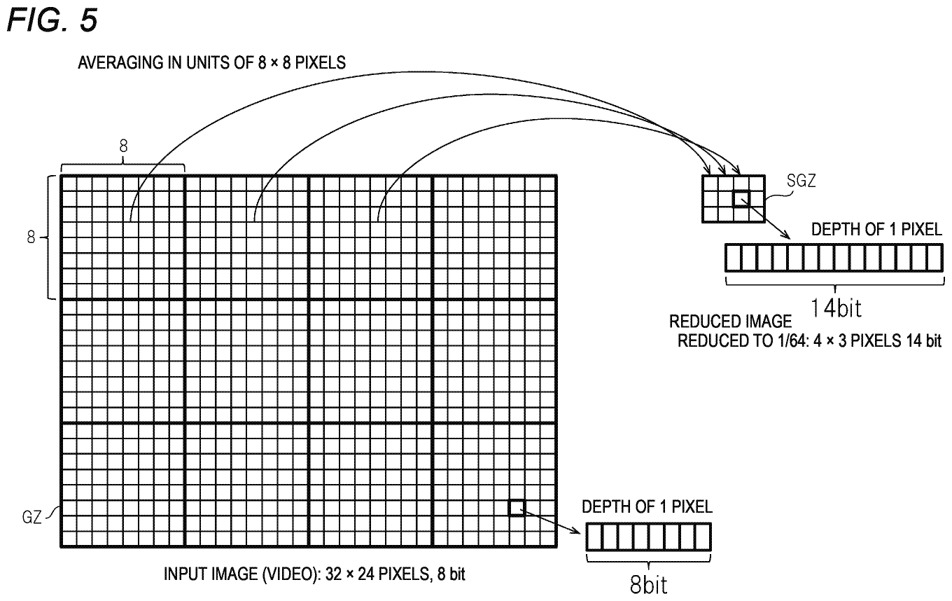

FIG. 5 is a diagram explaining the pixel addition and averaging of 8×8 pixels performed on the input image GZ. One pixel of the input image GZ has an information amount of a (a: a power of 2) bits (for example, 8 bits) (in other words, an information amount of gradations of 0 to 255). When a result of performing the pixel addition and averaging of 8×8 pixels (that is, 64 pixels) on the input image GZ is stored without the rounding processing, the number of bits capable of storing a data amount (=16320) of 255×“64”, which is the number of pixels subjected to the pixel addition and averaging, may be 14 bits (=0 to 16383) (16320<16383). That is, a pixel value after the pixel addition and averaging of 8×8 pixels can be recorded with 14 bits without the rounding processing. Here, in a case of a monochrome image, an information amount of one pixel after the pixel addition and averaging of 8×8 pixels is (a+b) bits (for example, 14 bits (=8+6)) (b: an integer of 2 or larger), whereas in a case of a color image, an information amount of one pixel (RGB pixels) after the pixel addition and averaging of 8×8 pixels is 42 bits (=(8+6)×3). That is, regardless of whether the image is a monochrome image or a color image, a value of b is an exponent (c) corresponding to a power of 2, which is the same as a product of 2\hat{ } {information amount (a) per pixel of the input image GZ} (=2<a>) and the number of pixels (8×8=64 pixels in the example described above) serving as a processing unit when performing the pixel addition and averaging, or an exponent (c+1) corresponding to the nearest power of 2, which is larger than the product.

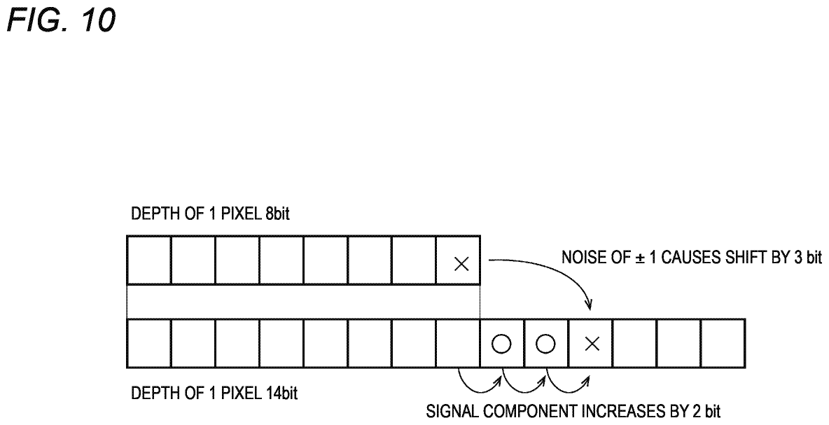

When the input image GZ is composed of S×T (S, T: positive integer, for example, S=32, T=24) pixels, the reduced image SGZ after the pixel addition and averaging of 8×8 pixels is reduced to 1/64 of the input image GZ that is the original image, and as a result, an information amount per pixel is expressed as 14 bits of 4×3 pixels (=(S×T)/N×M). In this case, among 14 bits per pixel, the upper 8 bits are integer values and the lower 6 bits are values after the decimal point (see FIG. 10).

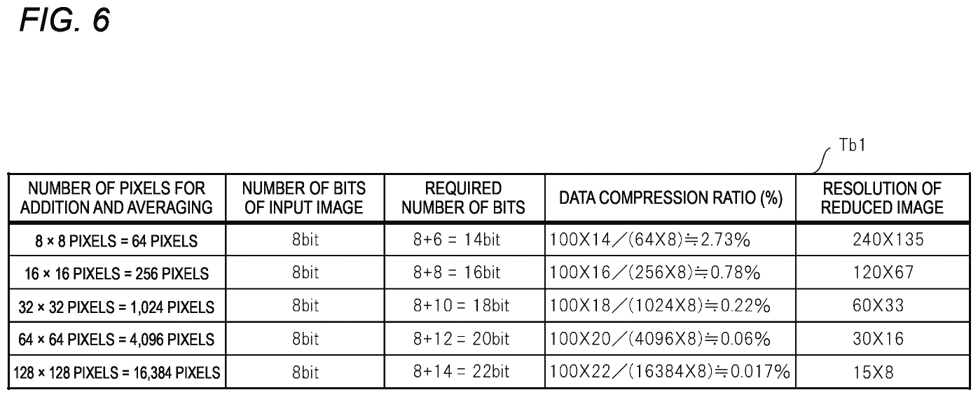

FIG. 6 is a diagram showing registered contents of an addition and averaging pixel number table Tb1. In the addition and averaging pixel number table Tb1, the number of bits (an information amount) required for one pixel after the pixel addition and averaging when the rounding processing is not performed is registered.

For example, when the pixel addition and averaging of 8×8 pixels is performed on an input image having a data amount of 8 bits per pixel, the number of bits (the information amount) required for one pixel is 14 (=8+6), and a data compression ratio is approximately 2.73%. When a resolution of the input image is 1920×1080 pixels of a full high-definition size, a resolution of the reduced image is 240×135 pixels, which is (⅛×8) times.

Similarly, when the pixel addition and averaging of 16×16 pixels is performed on an input image having the data amount of 8 bits per pixel, the number of bits (the information amount) required for one pixel is 16 (=8+8), and a data compression ratio is approximately 0.78%. When a resolution of the input image is 1920×1080 pixels, a resolution of the reduced image is 120×67 pixels, which is ( 1/16×16) times. Thereafter, similarly, when the pixel addition and averaging of 128×128 pixels is performed, the number of bits (the information amount) required for one pixel is 22 (=8+14), and a data compression ratio is approximately 0.017%. When a resolution of the input image is 1920×1080 pixels, a resolution of the reduced image is 15×8 pixels ( 1/128×128) times.

When a general processor stores data in the single-precision floating-point format, since a mantissa part is 23 bits, up to a pixel value after the pixel addition and averaging of 128×128 pixels, in which the number of bits (the information amount) required for one pixel is 22 bits, can be stored without the rounding processing.

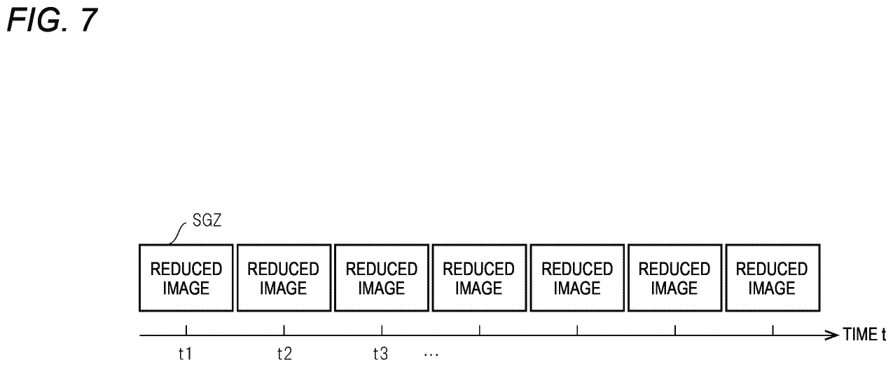

FIG. 7 is a diagram showing generation timings of the reduced image SGZ. The PC 30 performs the pixel addition and averaging on the input image GZ at predetermined timings t1, t2, t3 and so on along a time t direction for each frame image constituting the input moving image, and generates the reduced image SGZ. A data size of each reduced image SGZ is reduced (compressed) in the spatial direction, but is not reduced in the time direction (in other words, the reduced image SGZ is not generated by thinning out data timewisely), and the reduced image SGZ holds information indicating a minute change.

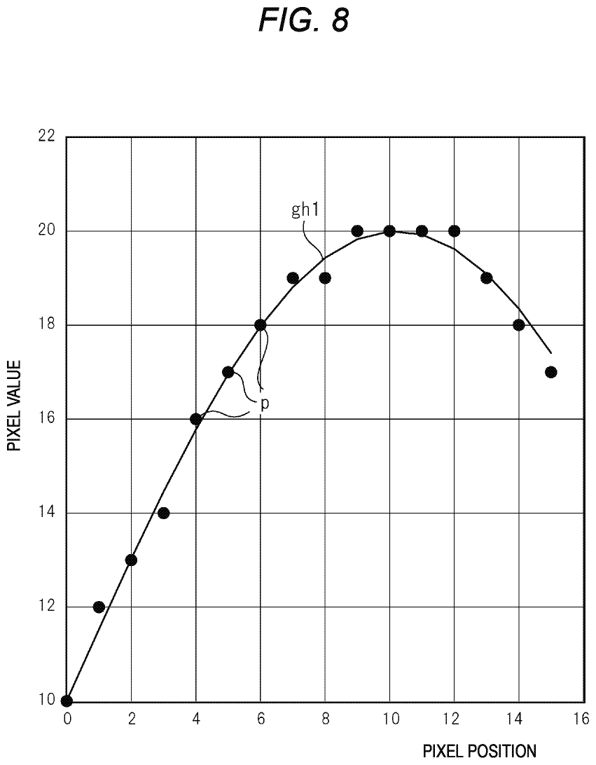

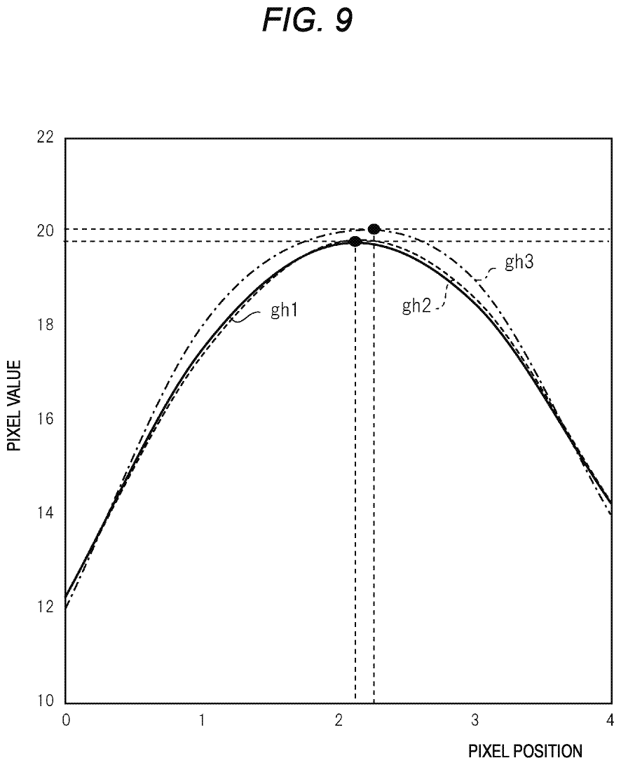

Here, an effect in a case where the rounding processing is not performed will be described in detail. FIG. 8 is a graph showing pixel value data of the input image GZ. FIG. 9 is a graph showing the pixel value data on which the rounding processing is not performed and the pixel value data on which the rounding processing is performed in the pixel addition and averaging. In each graph, a vertical axis represents a pixel value, and a horizontal axis represents a pixel position in a predetermined line of an input image.

Each point p in the graph of FIG. 8 represents each pixel value of the input image GZ (in other words, raw data). A curve graph gh1 is a fitting curve (a curve of the raw data) before pixel addition and averaging of four pixels is performed, which is fitted to the pixel value of each point p that is an actual measurement value, by, for example, a least-squares method. A curve graph gh2 represents a curve of the pixel value when the pixel addition and averaging of four pixels without the rounding processing is performed on the pixel value of each point p. A curve graph gh3 represents a curve of the pixel value when the pixel addition and averaging with the rounding processing is performed.

The curve graph gh2 draws a curve approximate to the curve graph gh1. In particular, peak positions of the curve graph gh2 and the curve graph gh1 coincide with each other. On the other hand, the curve graph gh3 draws a curve slightly deviated from the curve graph gh1. In particular, peak positions of the curve graph gh3 and the curve graph gh1 do not coincide with each other and are deviated from each other.

Therefore, when the sensing processing (for example, the motion detection) is performed using the curve graph gh3, since the peak position is shifted from each pixel value of the input image GZ (in other words, the raw data) in data obtained by performing the pixel addition and averaging with the rounding processing, an error may occur and an accurate motion position may not be detected. In contrast, in the data obtained by performing the pixel addition and averaging of four pixels without the rounding process, since the peak position coincides with each pixel value of the input image GZ (in other words, the raw data), the motion position can be accurately detected in the sensing processing.

FIG. 10 is a diagram explaining an effective component of a pixel signal when the pixel addition and averaging is performed without the rounding processing. Here, the image captured by the camera 10 includes optical shot noise (in other words, photon noise) caused by a solid-state imaging element (an image sensor) such as a CCD or a CMOS. The photon noise is generated when photons that jump in from a celestial body in outer space are detected by the image sensor. The optical shot noise has a characteristic that a noise amount is 1/N<(1/2)>times when pixel values are averaged and the number of pixels used for averaging is N.

For example, when the pixel addition and averaging of 8×8 pixels is performed, the noise amount is ⅛ times. Therefore, a noise component of the least significant bit (for example, noise of ±1) (indicated by x in the drawing) of 8-bit data is shifted to a lower side by three bits. When the noise component is shifted to the lower side by three bits, the effective component of the pixel signal (indicated by a circle in the drawing) increases by the lower two bits. That is, by performing the pixel addition and averaging without the rounding processing, the pixel signal can be restored with high accuracy.

Similarly, when the pixel addition and averaging of 16×16 pixels is performed, the noise amount is 1/16 times. Therefore, the noise of the least significant bit is shifted to the lower side by four bits. When the noise component is shifted to the lower level by four bits, the effective component of the pixel signal increases by the lower three bits. Therefore, the pixel signal can be restored with higher accuracy.

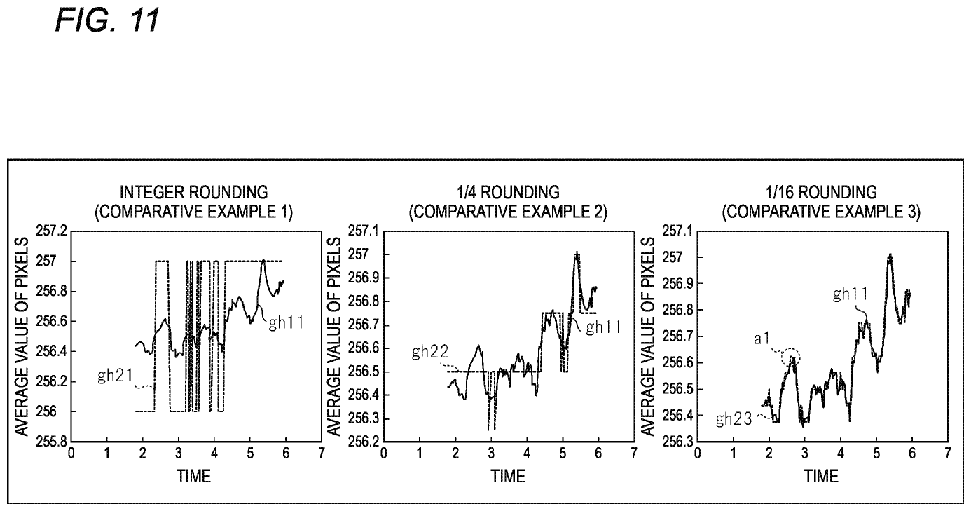

FIG. 11 is a graph showing image value data after the pixel addition and averaging with the rounding processing and the pixel value data after the pixel addition and averaging without the rounding processing according to the present embodiment in each of Comparative Example 1, Comparative Example 2 and Comparative Example 3. A curve graph gh210 according to Comparative Example 1 represents a graph after performing the pixel addition and averaging of 128×128 pixels with the rounding processing (integer rounding). The curve graph gh21 according to Comparative Example 1 hardly represents a minute change in the pixel value data.

A curve graph gh22 according to Comparative Example 2 represents a graph obtained by performing the pixel addition and averaging of four pixels without the rounding processing after performing the pixel addition and averaging of 64×64 pixels with the rounding processing. The curve graph gh22 according to Comparative Example 2 represents a tendency of the pixel value data, but does not accurately reflect a value of the pixel value data.

A curve graph gh23 according to Comparative Example 3 represents a graph obtained by performing the addition and averaging of 16 pixels without the rounding processing after performing the pixel addition and averaging of 32×32 pixels with the rounding processing. The curve graph gh23 according to Comparative Example 3 is similar to a curve graph gh11 according to the present embodiment as compared with Comparative Example 1 and Comparative Example 2, and reflects the pixel value data accurately to some extent. However, a peak position is deviated in a region indicated by a symbol al.

In this way, the curve graphs gh21, gh22, gh23 of Comparative Example 1, Comparative Example 2 and Comparative Example 3 do not accurately reflect the pixel value data as in the curve graph gh11 of the pixel value data after the pixel addition and averaging without the rounding process according to the present embodiment.

Next, an operation of the image processing system 5 according to the first embodiment will be described.

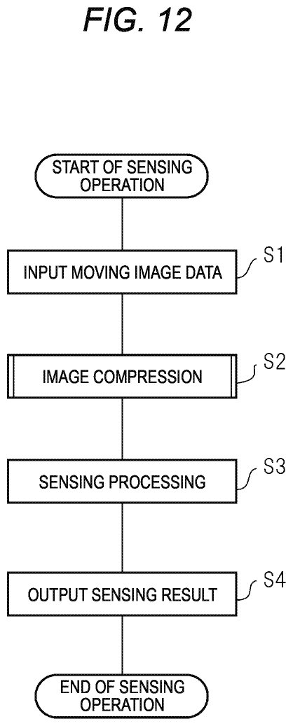

FIG. 12 is a flowchart showing a sensing operation procedure of the image processing system 5 according to the first embodiment. Processing shown in FIG. 12 is executed by, for example, the PC 30.

In FIG. 12, the processor 31 of the PC 30 inputs moving image data captured by the camera 10 (that is, data of each frame image constituting the moving image data) via the image input interface 36 (S1). The moving image captured by the camera 10 is, for example, an image at a frame rate of 60 fps. The image of each frame unit is input to the PC 30 as an input image (the original image) GZ.

The averaging processing unit 31a of the processor 31 performs pixel addition and averaging on the input image GZ. The reduced image generating unit 31b of the processor 31 generates the reduced image SGZ of a specific size (S2). Here, the specific size is represented by N×M pixels, and is, for example, 8×8 pixels (N=M=8).

The sensing processing unit 31c of the processor 31 performs sensing processing for determining presence or absence of a change in the input image GZ based on the reduced image SGZ (S3). The processor 31 outputs a result of the sensing processing (S4). As a result of the sensing processing, for example, the processor 31 may superimpose and display a marker on the captured image captured by the camera 10 such that a minute change appearing in the captured image is easily visually recognized. When motion information appearing in the captured image moves as a result of the sensing processing, the processor 31 may control the control device 40 so as to match a movement destination.

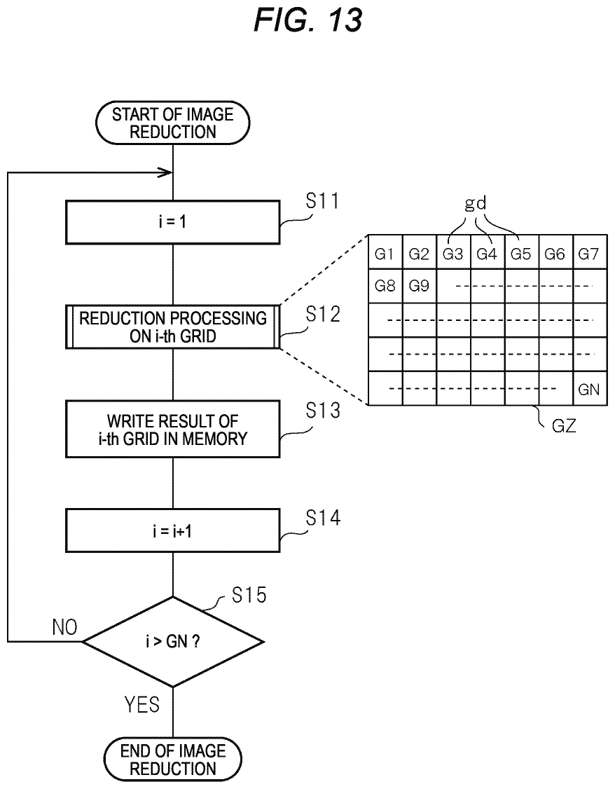

FIG. 13 is a flowchart showing an image reduction processing procedure in step S2. Here, a case where a reduced image is generated by performing the pixel addition and averaging of N×M pixels is shown. The averaging processing unit 31a of the processor 31 divides the input image GZ in grid units. A grid gd is a region obtained by dividing the input image GZ in units of k×1 (k, 1: an integer of 2 or larger) pixels. Each divided grid gd is represented by a grid number (G1, G2 to GN). Here, a case where the input image GZ is divided into grids gd in units of k (for example, 5)×1 (for example, 7) pixels and the maximum value GN of the grid number is 35 is shown.

The processor 31 sets a variable i representing the grid number to an initial value 1 (S11). The processor 31 performs reduction processing on the i-th grid gd (S12). Details of the reduction processing will be described later. The processor 31 writes a result of the reduction processing of the i-th grid gd in the memory 32 (S13).

The processor 31 increases the variable i by a value 1 (S14). The processor 31 determines whether the variable i exceeds the maximum value GN of the grid number (S15). When the variable i does not exceed the maximum value GN of the grid number (S15, NO), the processing of the processor 31 returns to step S12, and the processor 31 repeats the same processing for the next grid gd. On the other hand, when the variable i exceeds the maximum value GN of the grid number in step S15 (S15, YES), that is, when the reduction processing is performed on all the grids gd, the processor 31 ends the processing shown in FIG. 13.

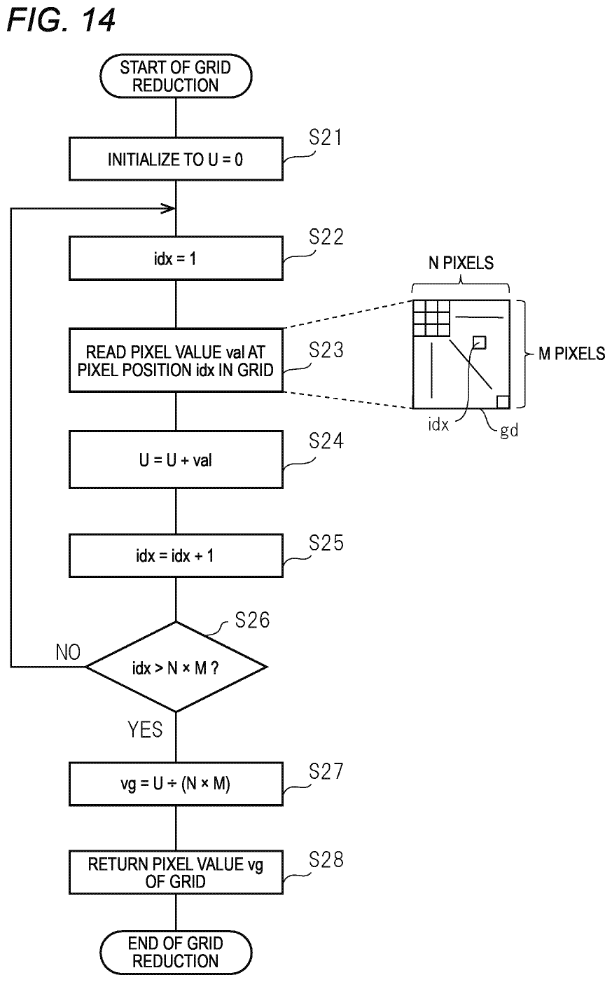

FIG. 14 is a flowchart showing a grid unit reduction processing procedure in step S12. The grid gd includes N×M pixels. N, M may be a power of 2 or may not be a power of 2. For example, N×M may be 10×10, 50×50 or the like. Each pixel in the grid is designated by a variable idx of a pixel position serving as an address. The processor 31 sets a grid value U to an initial value 0 (S21). The processor 31 sets the variable idx representing the pixel position in the grid to the value 1 (S22). The processor 31 reads a pixel value val at the pixel position of the variable idx (S23). The processor 31 adds the pixel value val to the grid value U (S24).

The processor 31 increases the variable idx by the value 1 (S25). The processor 31 determines whether the variable idx exceeds a value N×M (S26). When the variable idx does not exceed the value N×M (S26, NO), the processing of the processor 31 returns to step S23, and the processor 31 repeats the same processing for the next grid.

On the other hand, when the variable idx exceeds the value N×M in step S26 (S26, YES), the processor 31 divides the grid value U after the pixel addition and averaging of the N×M pixels by N×M according to Equation (1), and calculates a pixel value vg of the grid (S27).

[Equation 1]

vg=U÷(N×M) (1)

The processor 31 returns the pixel value vg of the grid after the pixel addition and averaging of the N×M pixels (that is, a calculation result of Equation (1)) to the original processing as the result of the reduction processing of the grid gd (S28). Thereafter, the processor 31 ends the grid unit reduction processing and returns to the original processing.

Here, when the reduced image after the addition and averaging of the N×M pixels as the specific size is generated, the N×M pixels are fixed or freely set (for example, to 8×8 pixels). The specific size may be set to a size suitable for a sensing target by the processor 31.

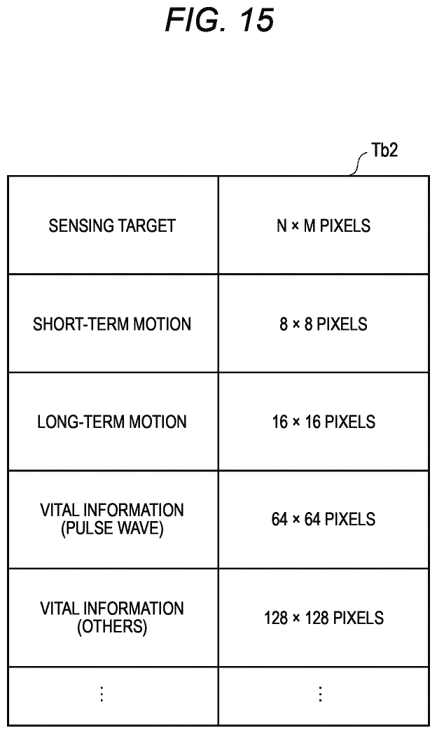

FIG. 15 is a diagram showing registered contents of a specific size selection table Tb2 indicating the specific size corresponding to the sensing target. The specific size selection table Tb2 is registered in the memory 32 in advance, and the registered contents can be referred to by the processor 31.

In the specific size selection table Tb2, when the sensing target is a short-term motion, 8×8 pixels are registered as N×M pixels representing the specific size. When the sensing target is a long-term motion (a slow motion), for example, 16×16 pixels are registered. When the sensing target is a pulse wave as vital information, 64×64 pixels are registered. When the sensing target is other vital information, 128×128 pixels are registered.

For example, when the sensing target is input from the user via the operation unit 34, the processor 31 may refer to the specific size selection table Tb2 and select the specific size corresponding to the sensing target in the processing of step S2. Accordingly, a change due to an image of a sensing target can be accurately captured.

In this way, in the image processing system 5 according to the first embodiment, the PC 30 performs the pixel addition and averaging on the input image from the camera 10 in units of N×M pixels, and holds a value of a decimal point level when the rounding processing (that is, the integer conversion processing) is not performed on the pixel value data obtained by the averaging processing, that is, when a resolution in the spatial direction is reduced and an amount of image information is compressed. By not performing the rounding processing on the value of the decimal point level, it is possible to compress the amount of the image information while holding the information having a minute change in the time direction (data necessary for image sensing). Therefore, the PC 30 can reduce an amount of processing by the sensing processing and an amount of memory required for data storage.

As described above, in the image processing system 5 according to the present embodiment, the PC 30 includes the averaging processing unit 31a and the reduced image generating unit 31b. The averaging processing unit 31a averages the input image GZ composed of 32×24 pixels having an information amount of 8 bits per pixel, in units of 8×8 pixels (N×M pixels (N, M: an integer of 2 or larger)) in the spatial direction for each grid composed of 64 pixels (one pixel or a plurality of pixels), for example. The reduced image generating unit 31b defines an averaging result in units of 8×8 pixels (N×M pixels) for each pixel or grid by an information amount of (8+6) bits per pixel, and generates the reduced image SGZ composed of 32×24/8×8 pixels having the information amount of (8+6) bits per pixel. Here, b is 6 (an exponent c (c: a positive integer) of a power value of 2 close to (N×M), or (c+1)). The sensing processing unit 31c senses motion information or biological information of an object using the reduced image SGZ.

Accordingly, the image processing system 5 can effectively compress each image (the frame image) constituting the moving image input from the camera 10 and reduce the data size. The image processing system 5 can prevent deterioration of detection accuracy of presence or absence of the motion information or the biological information of the object in the compressed image (in other words, accuracy of the sensing processing performed after the compression processing) while effectively compressing the input image.

The PC 30 further includes the sensing processing unit 31c that senses the motion information or the biological information of the object using the reduced image SGZ. Every time the input image GZ is input, the reduced image generating unit 31b outputs the reduced image SGZ generated corresponding to the input image GZ to the sensing processing unit 31c. Accordingly, the PC 30 can detect a change in the motion information and the biological information of the subject in real time based on the moving image captured by the camera 10.

The averaging processing unit 31a sends an averaging result to the reduced image generating unit 31b without performing the rounding processing. Accordingly, when the PC 30 reduces the size in the spatial direction to generate a reduced image and reduce the data amount, the PC 30 does not perform the rounding processing on the data after the decimal point, thereby preventing the information in the time direction from being lost. Accordingly, the PC 30 can accurately capture the minute change in the input image.

The averaging processing unit 31a acquires type information of the sensing of the motion information or the biological information of the object using the reduced image SGZ, selects a value of N×M according to the type information, and performs averaging in units of N×M pixels. Accordingly, the averaging processing unit 31a can perform the sensing using a reduced image suitable for a sensing target (the type information), and can accurately capture a minute change of the sensing target.

The PC 30 further includes the sensing processing unit 31c that senses the motion information and the biological information of the object using the reduced image SGZ. The averaging processing unit 31a selects a value of 8×8 (a first N×M) corresponding to sensing of the motion information and a value of 64×64 (at least one second N×M) corresponding to sensing of the biological information, and performs averaging in units of N×M pixels using the respective values of N×M. Accordingly, the PC 30 can perform the sensing using a reduced image suitable for the motion information of the object. In addition, the PC 30 can perform the sensing using a reduced image suitable for the biological information.

The averaging processing unit 31a averages the input image in units of a plurality of N×M pixels having different values of M, N. The reduced image generating unit 31b generates a plurality of reduced images SGZ1, SGZ2 and so on by averaging a plurality of N×M pixel units. As a result of performing the sensing using the plurality of reduced images SGZ1, SGZ2 and so on, the sensing processing unit 31c selects a reduced image suitable for sensing the motion formation or the biological information of the object. Accordingly, even if the sensing target is unknown and a reduced image suitable for the sensing target is not known in advance, the sensing can be performed with an optimum reduced image by actually testing the sensing using generated reduced images.

### First Modification of First Embodiment

Next, a first modification of the first embodiment will be described. A configuration of an image processing system according to the first modification of the first embodiment is the same as that of the image processing system 5 according to the first embodiment.

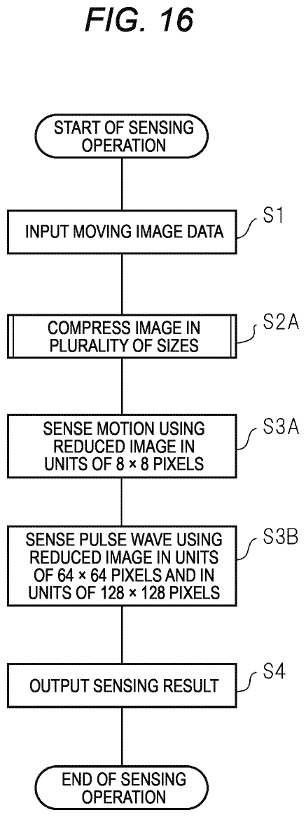

FIG. 16 is a flowchart showing a sensing operation procedure of the image processing system 5 according to the first modification of the first embodiment. The same step processing as the step processing shown in FIG. 12 is denoted by the same step number, description thereof will be simplified or omitted, and different contents will be described.

In FIG. 16, the processor 31 inputs moving image data captured by the camera 10 via the image input interface 36 (S1).

The averaging processing unit 31a of the processor 31 compresses an input image as an original image in a plurality of sizes, and the reduced image generating unit 31b generates a plurality of reduced images of each size (S2A). When the reduced images of a plurality of sizes are generated, it is desirable that the plurality of sizes include at least 8×8 pixels, 64×64 pixels and 128×128 pixels.

The sensing processing unit 31c of the processor 31 performs sensing of a motion as a change in the input image (an example of motion detection processing) using, for example, the reduced image in units of 8×8 pixels (S3A). Further, the processor 31 performs sensing of a pulse wave as a change in the input image (an example of pulse wave detection processing) using the reduced image in units of 64×64 pixels and in units of 128×128 pixels (S3B). The processor 31 outputs a result of the detection processing (S4).

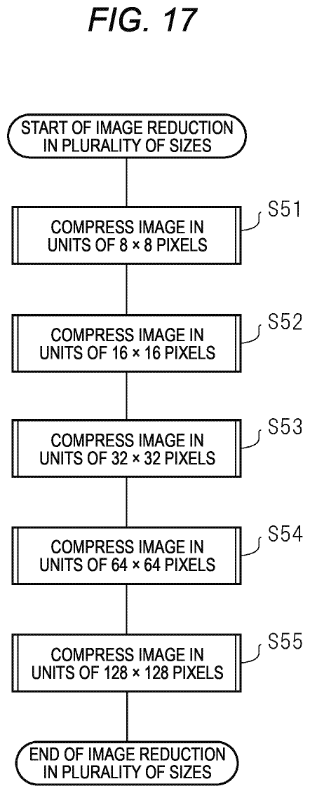

FIG. 17 is a flowchart showing a procedure for generating the reduced images in the plurality of sizes in step S2A.

In FIG. 17, the averaging processing unit 31a compresses the input image as an original image, and the reduced image generating unit 31b generates a reduced image in units of 8×8 pixels (S51). The averaging processing unit 31a compresses the input image as an original image, and the reduced image generating unit 31b generates a reduced image in units of 16×16 pixels (S52). The averaging processing unit 31a compresses the input image as an original image, and the reduced image generating unit 31b generates a reduced image in units of 32×32 pixels (S53). The averaging processing unit 31a compresses the input image as an original image, and the reduced image generating unit 31b generates a reduced image in units of 64×64 pixels (S54). The averaging processing unit 31a compresses the input image as an original image, and the reduced image generating unit 31b generates a reduced image in units of 128×128 pixels (S55). Thereafter, the processor 31 returns to the original processing.

In this way, the averaging processing unit 31a averages the input image in units of a plurality of N×M pixels having different values of M, N. The reduced image generating unit 31b generates a plurality of reduced images SGZ1, SGZ2 and so on by averaging a plurality of N×M pixel units. As a result of performing sensing using the plurality of reduced images SGZ1, SGZ2, and so on, the sensing processing unit 31c selects a reduced image suitable for sensing motion information or biological information of an object, and thereafter performs sensing processing using the selected reduced image. Therefore, even if a sensing target is unknown and a reduced image suitable for the sensing target is not known in advance, the sensing processing can be performed with an optimum reduced image by actually testing the sensing using all the reduced images.

When addition and averaging is performed with a predetermined number of pixels, the processor may perform the addition and averaging of the number of pixels in a stepwise manner. For example, when the processor 31 performs the addition and averaging on the input image in units of 16×16 pixels, the processor 31 may first perform the pixel addition and averaging on the input image in units of 8×8 pixels, and perform the pixel addition and averaging on the reduced image that is the averaging result in units of 2×2 pixels. Similarly, when the processor performs the pixel addition and averaging on the input image in units of 32×32 pixels, the processor may first perform the pixel addition and averaging on the input image in units of 16×16 pixels, and perform the pixel addition and averaging on the reduced image that is the averaging result in units of 2×2 pixels.

That is, when averaging the input image in units of N×M pixels for each grid, the processor may sequentially repeat processing of averaging the input image in units of pixels of one set of first factors×second factors by using a predetermined number of first factors obtained by decomposing M into a product form and a predetermined number of second factors obtained by decomposing N into a product form, and averaging the averaging result in units of pixels of the remaining one set of first factors×the other second factors until all of the predetermined number of first factors and the predetermined number of second factors are used.

In this way, the same averaging result can be obtained as in a case where the addition and averaging is repeatedly performed in units of a small number of pixels and the addition and averaging is performed in units of a large number of pixels at one time, and an amount of data processing can be reduced.

### Second Modification of First Embodiment

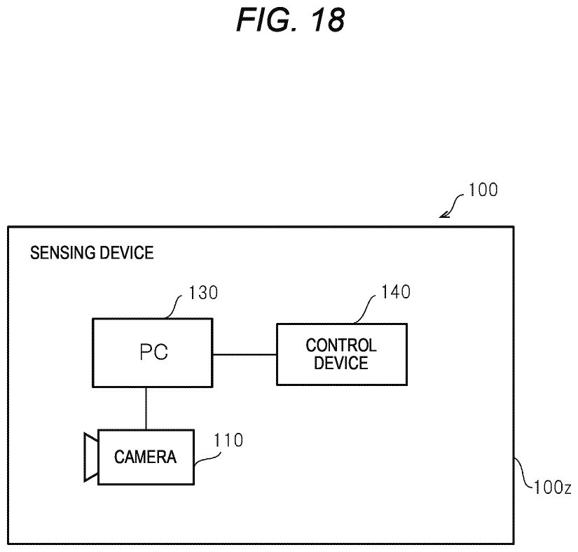

In the first embodiment, the camera 10, the PC 30 and the control device 40 are configured as separate devices. In a second modification of the first embodiment, the camera 10, the PC 30 and the control device 40 may be accommodated in the same housing and configured as an integrated sensing device. FIG. 18 is a diagram showing a configuration of an integrated sensing device 100. The integrated sensing device 100 includes a camera 110, a PC 130 and a control device 140 accommodated in a housing 100z. The camera 110, the PC 130 and the control device 140 have functional configurations the same as the camera 10, the PC 30 and the control device 40 according to the above-described embodiment, respectively. As an example, when the integrated sensing device 100 is applied to an air conditioner, the camera 110 is disposed on a front surface of a housing of the air conditioner. The PC 130 is built in the housing, generates a reduced image using each frame image of the moving image captured by the camera 110 as an input image, performs sensing processing using the reduced image, and outputs a sensing processing result to the control device 140. In a case of the integrated sensing device 100, a display unit and an operation unit of the PC may be omitted. The control device 140 controls an operation according to an instruction from the PC 130 based on the sensing processing result. When the control device 140 is an air conditioner main body, the control device 140 adjusts a wind direction and an air volume.

In the case of the integrated sensing device 100, an image processing system can be designed in a compact manner. When the sensing device 100 is portable, it is possible to move the sensing device 100 to any place and perform installation adjustment. The sensing device 100 can be used even in a place where there is no network environment.

Although various embodiments have been described above with reference to the drawings, it is needless to say that the present disclosure is not limited to such examples. It will be apparent to those skilled in the art that various alterations, modifications, substitutions, additions, deletions and equivalents can be conceived within the scope of the claims, and it should be understood that such changes also belong to the technical scope of the present disclosure. Components in the above-described embodiments may be combined optionally within a range not departing from the spirit of the invention.

For example, in the above-described embodiment, for example, a video of 60 fps is exemplified as a moving image, but a time-continuous frame image, for example, about five continuous still images per second may be used.

The image processing system can be used for sports, animals, watching, drive recorders, intersection monitoring, moving images, rehabilitation, microscopes and the like, in addition to the above embodiments. In sports, for example, the image processing system can be used for motion check, form check or the like. In animals, the image processing system can be used for an activity area, a flow line or the like. In watching, the image processing system can be used for a vital sign, an amount of activity, rolling over during sleep or the like in a baby or an elderly home. In drive recorders, the image processing system can be used to detect a motion around a vehicle shown in a captured video. In intersection monitoring, the image processing system can be used for a traffic volume, a flow line and an amount of signal disregard. In moving images, the image processing system can be used to extract a feature included in a frame amount. In rehabilitation, the image processing system can be used for confirmation of an effect from a vital sign, a motion or the like. In microscopes, the image processing system can be used for automatic detection of a slow motion, or the like.

The present disclosure is useful as an image processing device, an image processing method and an image processing system capable of, in image processing, effectively compressing an input image to reduce a data size and preventing deterioration in detection accuracy of presence or absence of motion information or biological information of an object in the compressed image.

What is claimed is:

1. An image processing device comprising:
a memory that stores instructions; and
a processor that, when executing the instructions stored in the memory, performs a process, wherein the process including:
averaging an input image in units of N×M pixels (N, M: an integer of 2 or larger) in a spatial direction for each grid composed of one pixel or a plurality of pixels, the input image being composed of (S×T) pixels (S, T: a positive integer) having an information amount of a (a: a power of 2) bits per pixel; and
defining an averaging result in units of N×M pixels for each pixel or grid by an information amount of (a+b) bits per pixel (b: an integer of 2 or larger) and generating a reduced image composed of (S×T)/(N×M) pixels having the information amount of (a+b) bits per pixel,
wherein a value of b is an exponent c (c: a positive integer) of a power value of 2 close to (N×M), or (c+1).

2. The image processing device according to claim 1, wherein the process further including:
sensing motion information or biological information of an object using the reduced image,
wherein the reduced image generated corresponding to the input image is output by the processor to the sensing processing unit each time the input image is input.

3. The image processing device according to claim 1,
wherein the averaging result by the information amount of (a+b) bits per pixel is defined by the processor without performing rounding processing on the averaging result.

4. The image processing device according to claim 1,
wherein type information of sensing of motion information or biological information of an object using the reduced image is acquired, a value of (N×M) according to the type information is selected, and averaging in units of (N×M) pixels is performed by the processor.

5. The image processing device according to claim 1, wherein the process further including:
sensing motion information and biological information of an object using the reduced image,
wherein a value of a first (N×M) corresponding to sensing of the motion information and a value of at least one second value (N×M) corresponding to sensing of the biological information are selected, and averaging in units of (N×M) pixels using the respective values of (N×M) is performed by the processor.

6. The image processing device according to claim 2,
wherein the input image in units of N×M pixels in a plurality of pairs having different values of M, N, is averaged using the plurality of pairs;
wherein reduced images whose number is the same as the number of pairs obtained by averaging the plurality of pairs in units of N×M pixels are generated by the processor; and
wherein a reduced image suitable for sensing the motion information or the biological information of the object is selected by the processor based on a result of performing sensing using the reduced images whose number is the same as the number of the pairs.

7. An image processing method in an image processing device, the image processing method comprising:
averaging an input image in units of N×M pixels (N, M: an integer of 2 or larger) in a spatial direction for each grid composed of one pixel or a plurality of pixels, the input image being composed of (S×T) pixels (S, T: a positive integer) having an information amount of a (a: a power of 2) bits per pixel; and
defining an averaging result in units of N×M pixels for each pixel or grid by an information amount of (a+b) bits per pixel (b: an integer of 2 or larger) and generating a reduced image composed of (S×T)/(N×M) pixels having the information amount of (a+b) bits per pixel,
wherein a value of b is an exponent c (c: a positive integer) of a power value of 2 close to (N×M), or (c+1).

8. An image processing system in which an image processing device and a sensing device are connected so as to communicate with each other,
wherein the image processing device is configured to average an input image in units of N×M pixels (N, M:
an integer of 2 or larger) in a spatial direction for each grid composed of one pixel or a plurality of pixels, the input image being composed of (S×T) pixels (S, T: a positive integer) having an information amount of a (a: a power of 2) bits per pixel; and
is configured to define an averaging result in units of N×M pixels for each pixel or grid by an information amount of (a+b) bits per pixel (b: an integer of 2 or larger), generates a reduced image composed of (S×T)/(N×M) pixels having the information amount of (a+b) bits per pixel, and sends the reduced image to the sensing device;
wherein the sensing device is configured to sense motion information or biological information of an object using the reduced image sent from the image processing device; and
wherein a value of b is an exponent c (c: a positive integer) of a power value of 2 close to (N×M), or (c+1).
